(19) United States
(12) Patent Application Publication
(10) Pub. No.: US 2004/0138315 A1
(43) Pub. Date: Jul. 15, 2004

(54) Methods and compounds for inhibiting eicosanoid metabolism and platelet aggregation

(75) Inventor: Craig R. Travis, South Miami, FL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Immugen Pharmaceuticals, Inc., South Miami, FL (US)

(21) Appl. No.: 10/738,457

(22) Filed: Dec. 16, 2003

Related U.S. Application Data
(63) Continuation of application No. 09/967,353, filed on Sep. 28, 2001.
(60) Provisional application No. 60/236,408, filed on Sep. 28, 2000.

(51) Int. Cl. A61K 31/05; A01N 1/02

(52) U.S. Cl. 514/731; 435/2

(57) ABSTRACT

The invention provides a method for attenuating the aggregation and/or activation of blood platelets within a blood product. In accordance with this method, a cannabinoid or resorcinolic compound is introduced into the blood product under conditions sufficient to inhibit the aggregation and/or activation of blood platelets within the blood product. The invention also pertains to the use of a cannabinoid or resorcinolic compound to prepare a composition suitable for inhibiting the activation and/or aggregation of blood platelets and to such compositions. The invention also pertains to a method of selectively inhibiting COX-1 and thromboxane synthase within a cell or blood platelet.

### BACKGROUND OF THE INVENTION

Life-threatening vascular related disorders are mediated by a number of factors when the endothelial surface of blood vessels is exposed by spontaneous rupture or fissuring of an athermanous plaque leading to the formation of a vascular plug. This plug is comprised mainly of platelets, erythrocytes, leukocytes, thrombin and fibrin. Platelet aggregation is promoted by local and systemic factors such as adrenaline, adenosine diphosphate, prostaglandin E2, thromboxanes, calcium fluxes, and platelet receptor mediated events. A clinical prodrome exists as a result of the decreased blood flow through partially occluded vessels to the end organ resulting in ischemic pain or transient ischemic attacks affecting the central nervous system; further aggregation of platelets causes alterations in blood flow and the shearing forces exerted on red blood cells cause the release of adenosine diphosphate, which in turn causes further aggregation of platelets. Additional local factors such as calcium fluxes, concentration and activation of other hemostatic proteins also contribute the promotion of a clot. Eventually, the completion of a thrombus can lead to arterio-occlusive syndromes such as myocardial infarction, stroke and vascular occlusive syndromes such as arterial thrombosis within the splanchnic circulation and peripheral vasculature. The release of serotonin from platelets has also been a contributing factor to the pain of migraine headaches, which is a vasospastic disease process.

Pain can be the result of injury, inflammatory processes, surgical procedures, vaso-occlusive and vasospastic disorders, infection and distention of a hollow viscous which might result from a physical obstruction such as gallstone or renal calculus or occlusion of blood supply to the affected organ. Numerous local factors act to promote pain among these are the eiconosoids such as PGE2. Other inflammatory products such as cytokines and chemokines act in an autocrine and paracrine manner to promote a response to the injury. The resulting cascade of events usually results in increased pain and further inflammation. Pharmacological interventions of such conditions usually requires the use of analgesics, anti-inflammatory drugs, muscle relaxants, and when warranted, antibiotics. Surgical intervention is usually dictated by the circumstances and post-operative pain is typically managed by narcotic analgesics.

Arachidonic acid metabolism yields a variety of hormone like substances which include prostaglandins, prostacyclins, thromboxanes and leukotrienes which act in a local environment to mediate a variety of physiologic events including inflammatory response, fever and pain, the regulation of blood pressure, formation of a clot at the site of injury, the induction of labor and the regulation of the sleep/wake cycle.

Esterified arachidonic acid (5, 8, 11, 14-eicosatetraenoic acid) is stored primarily in cell membranes as a phosholipid which is released by three different mechanisms—phospholipase A2, phospholipase C and diacyglycerol lipase, each stimulated and regulated by varying autocrine and paracrine signaling molecules. The metabolism of arachidonate can proceed through a cyclic pathway (cyclooxygenase) which forms a cyclopentane ring which is characteristic of prostaglandins, or through a linear pathway which yields leukotrienes and HPETES. Two forms of the PG G/H synthase (prostaglandin H2 synthase; prostaglandin endoperoxide synthase) have been identified which are colloquially known as COX-1 and COX-2 (cyclooxygenase) enzymes that produce endoperoxides from arachidonic acid to serve as substrates for cell specific isomerases and synthases. Tissue specificity determines the end product of arachidonic acid metabolism. Platelets generally contain only COX-1 thromboxane, a potent vasoconstrictor and promoter of platelet aggregation. The inhibition of COX-1 in platelets prevents the formation of the endoperoxide substrates required for the synthesis of thromboxane, which not only inhibits platelet aggregation and vasoconstriction but also effects a redirection of eicosanoid metabolism to the production of prostacyclin in endothelial cells. Conversely, endothelial cells possess both COX isoenzymes, but COX-2 predominates to produce prostacyclin, which is inhibitory of platelet aggregation, leukocyte activation and adhesion, vascular smooth muscle contraction, migration and growth and cholesterol ester accumulation in vascular cells.

In light of the deleterious problems associated with blot platelet aggregation, there have been attempts to inhibit aggregation through chemical intervention using putative “platelet inhibitors,” aspirin being an archetype. In addition to the treatment of an acute coronary syndrome such as stable and unstable angina, acute myocardial infarction, non-Q wave MI by the prevention of further platelet aggregation, platelet inhibitors have been proposed to be employed for the prevention of arterio-occlusive syndromes such as stroke, claudication, during percutaneous coronary intervention, i.e. stents, and for the prevention of eiconosoid mediated vascular injury, focal ischemia and thrombosis associated with acute vascular rejection in organ transplantation. Additionally, such drugs have been proposed for use in prevention of thrombus formation in non-valvular atrial fibrillation, particularly in a low risk patient at risk for embolic stroke.

Despite the potential health benefits attributed to the inhibition of platelet aggregation, existing platelet inhibitory agents are less than ideal. For example, it is known that aspirin affects both COX-1 and COX-2 activity, it inhibits both thromboxane and prostacylin in platelets and endothelial cells respectively. However, since aspirin does not directly inhibit thromboxane synthase activity in platelets and monocyte/macrophages, thromboxane synthase remains intact to act on endothelium-derived endoperoxides PGG2 and PGH2 to allow for a significant transcellular thromboxane A2 biosynthesis. Indeed, aspirin shares a drawback with selective COX-2 inhibitors, both of which result in decreased production of prostacylin and its ability to contribute to the inhibition of platelet aggregation. Hence, the need for thromboxane A2 synthase inhibition in combination with selective COX-1 inhibition. Thus, there remains a need for additional reagents for attenuating the aggregation and/or activation of blood platelets. Moreover, in light of the problems attendant with non-selected COX-1 and -2 inhibition, there exists a need for reagents for selectively inhibiting COX-1.

### BRIEF SUMMARY OF THE INVENTION

The invention provides a method for attenuating the aggregation and/or activation of blood platelets within a blood product. In accordance with this method, a cannabinoid or resorcinolic compound is introduced into the blood product under conditions sufficient to inhibit the aggregation and/or activation of blood platelets within the blood product. The invention also pertains to the use of a cannabinoid or resorcinolic compound to prepare a composition suitable for inhibiting the activation and/or aggregation of blood platelets and to such compositions. The invention also pertains to a method of selectively COX-1 and thromboxane synthase within a cell or specialized tissue, such as a platelet.

The method and reagents of the present invention can be employed to help protect the supply of blood. In other applications, the method can be employed prophylactically or therapeutically within patients. These and other advantages of the present invention, as well as additional inventive features, will be apparent from the following detailed description.

### DETAILED DESCRIPTION OF THE INVENTION

The invention provides a method for attenuating the activation and/or aggregation of blood platelets within a blood product. In accordance with this method, a (i.e., at least one) cannabinoid or resorcinolic compound is introduced into the blood product under conditions sufficient to inhibit the activation and/or aggregation of blood platelets within the blood product.

Within the context of the invention, a “blood product” is any fraction derived from blood that contains platelets. Suitable blood product that can be treated in accordance with the invention include whole blood, plasma, packed red cells, etc. Where the blood product is ex vivo (typically donated blood and its fractions), the method can be employed to inhibit platelet aggregation within the product. Thus, the invention can be employed to help preserve such products for future use. In other embodiments, the blood product can be within an organ or tissue ex vivo (e.g., within the vasculature of the organ or tissue). In this aspect, the method can be used during organ transplantation or tissue engraftment to reduce or retard thrombus formation in the graft organ or tissue, and to otherwise promote successful transplantation. Of course, the method also can be used in vivo, in which it can be used as part of a regimen for controlling platelet activation and/or aggregation within suitable patients. In this regard, the method can assist in prophylaxis for conditions such as stroke, claudication, thrombus formation in non-valvular fibrillation, heart attacks, and other conditions that result from thrombosis within a patient. The method also can be employed therapeutically to address indications associated with eicosanoid metabolism such as acute inflammation, asthma and systemic anaphylaxis, transplant rejection, kidney pathophysiology and immune disorders, pain, inflammation, autoimmune diseases, ischemic conditions mediated by platelets, vascular conditions mediated by the expression of prostaglandins, thromboxanes and/or phospholipid metabolism. The method also can be used to treat other conditions such as migraine headache and variants, TIA (transient ischemic attacks), angina pectoris both stable and unstable and myocardial infarction. In other aspects, the method can be used adjunctively during surgical procedures, which includes surgical revascularization, for example, catheterization, or other invasive procedures performed on a patient to prevent unwanted clotting or thrombus formation at the site of invasion or on or within devices (e.g., catheters, stents, etc.) used in such procedures. The compounds also can be employed for inhibiting the peroxidation of LDL lipid.

In one embodiment, at least one compound introduced into the blood product can be a resorcinol derivative. Such compounds are advantageous for use in vivo as they generally exhibit low cytotoxicity (see, e.g., U.S. Pat. No. 5,859,067). Exemplary resorcinols can have the following formula:

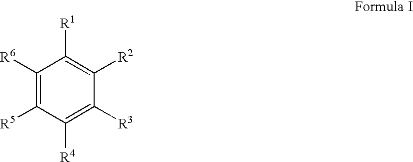

wherein,

R¹, R³, R⁵, and R⁶ can optionally be —COR¹, —COR³, —COR⁵, and/or —COR⁶, respectively, and preferably R³ is —COR³, and wherein R can otherwise be as follows:

R¹ is: a) H,

b) a C₁₋₄ alkyl group or ester thereof,

c) COOH,

d) OH,

e) a O—C₁₋₅ alkyl (preferably OCH₃) or alkanoyl, optionally substituted by mono- or di-methylamino or ethylamino groups,

f) a O—CO—C₃₋₁₀ alkyl group containing a carboxyl or amino group,

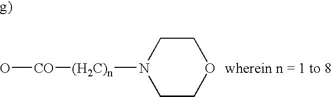

h) a p-aminobenzyl group or a C₁₋₇ aminoalkyl group or an organic or mineral acid addition salt thereof, an isocyanate or isothiocyanate derivative of the p-aminobenzyl or aminoalkyl group, a carboxyl terminated derivative of the aminoalkyl group having from 1 to 7 additional carbon atoms or a salt thereof, and an activated derivative of the carboxyl terminated derivative;

i) R¹ and R² comprise a substituent of the formula —O(CH₂)₃₋₅, wherein R¹ and R², together with the carbon atoms to which they are bonded, comprises a ring where at least one hydrogen atom thereof is optionally substituted with a halogen (e.g., fluorine, bromine, iodine, astatine);

j) a lactone (e.g., COCOH); or

k) CH(CH₃)CO₂H or —OCOCH₃

R² is: a) H, OH, COOH, or a halogen

b) C₁₋₆ carboxy or alkoxy group, or

c) R¹ and R² comprise a substituent of the formula —O(CH₂)₃₋₅, wherein R¹ and R², together with the carbon atoms to which they are bonded, comprises a ring where at least one hydrogen atom thereof is optionally substituted with a halogen.

R³ is: a) (W)ₘ-Y-(Z)ₙ, wherein

W is a C₅₋₁₂ straight or branched (preferably 1S′CH₃, 2R′CH₃ dimethyl) alkyl (e.g., -pentyl, -hexyl, -heptyl, -octyl, or -nonyl), alkenyl, alkynyl, group, or mixture thereof, optionally substituted with at least one halogen (e.g., halogen terminal group or even dihalogen),

Y is a bond, O, S, SO, SO₂, CO, NH, N(C₁₋₆ alkyl), or NCS,

Z is: i) a C₅₋₁₂ alkyl, alkenyl, alkynyl, group, or mixture thereof, optionally substituted with at least one halogen, optionally substituted with a terminal aromatic ring,

ii) CN₁₋₃, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different, or

iii) a phenyl or benzyl group, optionally substituted with halo, C₁₋₆ alkyl, C₁₋₆ alkoxy, C₁₋₆ alkylthio, CN, CF₃, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different, and wherein

m and n are the same or different, and each is either 0 or 1,

b) a C₅₋₁₂ alkyl or haloalkyl group, optionally substituted with a terminal aromatic ring, CN₁₋₃, NCS, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different, or

c) a C₅₋₁₂ alkene or alkyne group, optionally substituted with a halogen, dithiolene, terminal aromatic ring, CN₁₋₃, NCS, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different;

R⁴ is: a) H or halogen (preferably bromine)

b) OH, or

c) C₁₋₆ alkoxyl or carboxyl;

R⁵ is a)H,

b) a C₁₋₄ alkyl group,

c) COOH,

d) OH, or OCH₃,

e) a O—C₁₋₅ alkyl (ether) or alkanoyl, optionally substituted with at least one mono- or di-methylamino or ethylamino group, or

f) a lactone; and

R⁶ is: a) H or OH;

b) C₁₋₄ alkyl (preferably ethyl), alkenyl, alkynyl, group, or mixture thereof,

c) O—C₁₋₄ alkyl, alkenyl, alkynyl, group, or mixture thereof, or

d) a pryenyl, gerenyl, or farnesyl group, optionally substituted at any position with one or more halogens,

e) (W)ₘ-Y-(Z)ₙ, wherein

W is a C₅₋₁₂ alkyl, alkenyl, alkynyl, group, or mixture thereof, optionally substituted with at least one halogen,

Y is a bond, O, S, SO, SO₂, CO, NH, N(C₁₋₆ alkyl), or NCS,

Z is: i) a C₅₋₁₂ alkyl, alkenyl, alkynyl, group, or mixture thereof, optionally substituted with at least one halogen, optionally substituted with a terminal aromatic ring,

ii) CN₁₋₃, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different, or

iii) a phenyl or benzyl group, optionally substituted with halo, C₁₋₆ alkyl, C₁₋₆ alkoxy, C₁₋₆ alkylthio, CN, CF₃, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different, and wherein

m and n are the same or different, and each is either 0 or 1,

f) a C₅₋₁₂ alkyl or haloalkyl group, optionally substituted with a terminal aromatic ring, CN₁₋₃, NCS, CO₂H, or CO₂C₁₋₄ alkyl, CONH₂, CONHC₁₋₄alkyl, or CON(C₁₋₄ alkyl)₂, wherein each C₁₋₄ alkyl on the amide nitrogen can be the same or different,

g) a C₅₋₁₂ alkene or alkyne group, optionally substituted with a halogen, dithiolene, terminal aromatic ring, CN₁₋₃, NCS, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different, or

h) CH(CH₃)CO₂H, CH₂COOH, or —OCOCH₃.

Compounds according to Formula I preferably include a lactone, H, OH or OCH₃, —CH(CH₃)CO₂H, or —OCOCH₃ as R¹ substituents. Preferred substituents at R² are hydrogen, halogen (most preferably fluorine) hydroxyl, COOH, or methoxyl groups. Preferred substituents at R⁴ include H or a halogen (most preferably bromine). Preferred substituents at R⁵ include a lactone, H, OH, and OCH₃. Preferred substituents at R⁶ include H, OH, ethyl, CH(CH₃)CO₂H, CH₂COOH, and —OCOCH₃. Where compounds of formula I are included, preferably R⁶ is methyl or ethyl. A more preferred compound according to Formula I has hydroxyl substituents at R¹, R⁵, and a methyl substituent at R⁶; even more preferably, the compound has a third hydroxyl substituent at R². Preferred substituents at R³ are discussed elsewhere herein; however, the invention provides compounds according to Formula I, wherein R³ is:

a) (W)ₘ—Y-(Z)ₙ, wherein

W is a C₅₋₁₂ alkyl, alkenyl, alkynyl (e.g., 2′-ynyl, 3′-ynyl or 4′-ynyl), group, or mixture thereof, optionally substituted with at least one halogen,

Y is a bond, O, S, SO, SO₂, CO, NH, N(C₁₋₆ alkyl), or NCS,

Z is: i) a C₅₋₁₂ alkyl, alkenyl, alkynyl (e.g., 2′-ynyl, 3′-ynyl or 4′-ynyl), group, or mixture thereof, optionally substituted with at least one halogen, optionally substituted with a terminal aromatic ring,

ii) CN₁₋₃, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different, or

iii) a phenyl or benzyl group, optionally substituted with halo, C₁₋₆ alkyl, C₁₋₆ alkoxy, C₁₋₆ alkylthio, CN, CF₃, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different,

wherein at least one of W and Z includes a branched chain and wherein m and n are the same or different, and each is either 0 or 1,

b) a terminally-branched (e.g., terminal dimethyl) C₅₋₁₂ alkyl or haloalkyl group, optionally substituted with a terminal aromatic ring, CN₁₋₃, NCS, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different, or

c) a terminally-branched C₅₋₁₂ alkene or alkyne group, optionally substituted with a halogen, dithiolene, terminal aromatic ring, CN₁₋₃, NCS, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different.

Particularly preferred R³ substituents include C₅-C₁₂ alkynes, and particularly preferred groups also include di- or tri-methyl terminal groups. A most preferred substituent at R³ is a dimethylheptyl, particularly 1′S, 2′SR, and also preferably with terminal halogen (or dihalogen) substituents, and another preferred substituent is 5,5-diimethyl hex(1-ene)(3-yne)yl (e.g., compound ii). While any such compounds can be included within the composition in accordance with the inventive method, some preferred compounds are as follows:

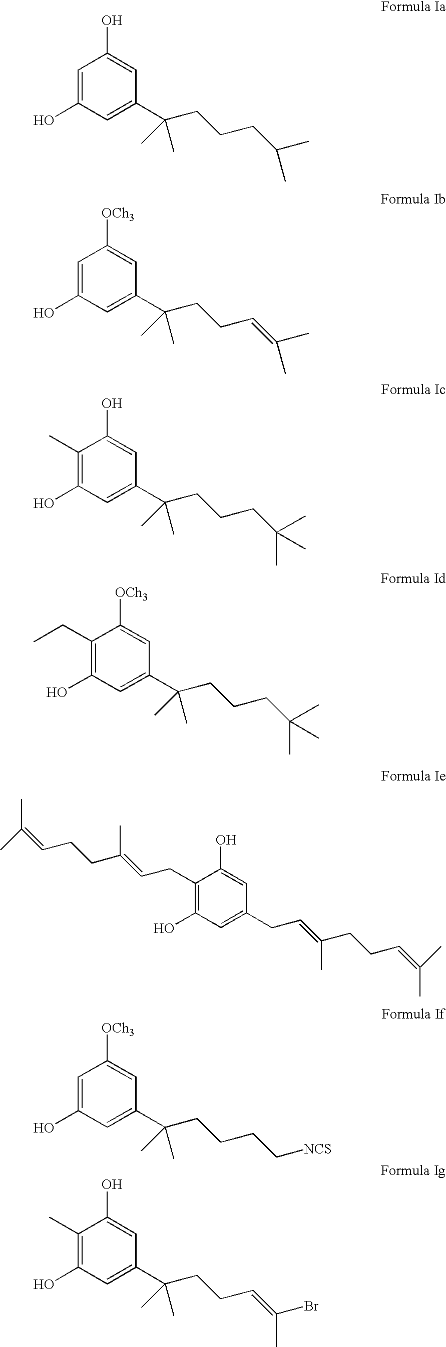

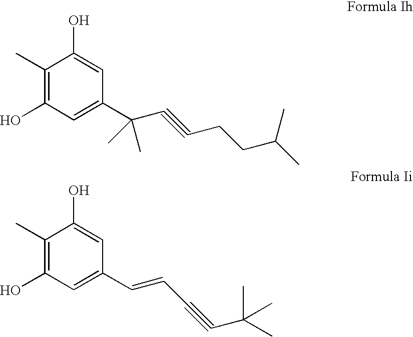

In another embodiment, at least one compound for introduction into the blood product can be a cannabinol derivative having the following formula:

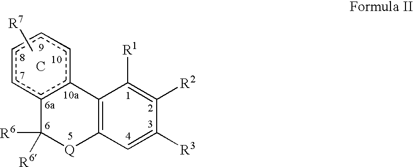

wherein,

R¹ is: a) H.

b) a C₁₋₄ alkyl group or ester thereof,

c) COOH,

d) OH,

e) a O—C₁₋₅ alkyl (preferably OCH₃) or alkanoyl, optionally substituted by mono- or di-methyl amino or ethyl amino groups,

f) a O—CO—C₃₋₁₀ alkyl group containing a carboxyl or amino group,

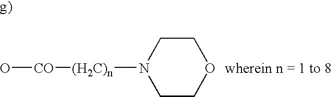

h) a p-aminobenzyl group or a C₁₋₇ aminoalkyl group or an organic or mineral acid addition salt thereof, an isocyanate or isothiocyanate derivative of the p-aminobenzyl or aminoalkyl group, a carboxyl terminated derivative of the aminoalkyl group having from 1 to 7 additional carbon atoms or a salt thereof, and an activated derivative of the carboxyl terminated derivative;

i) R¹ and R² comprise a substituent of the formula —O(CH₂)₃₋₅, wherein R¹ and R², together with the carbon atoms to which they are bonded, comprises a ring where at least one hydrogen atom thereof is optionally substituted with a halogen;

j) a lactone (e.g., COCOH); or

k) CH(CH₃)CO₂H or —OCOCH₃

R² is: a) H, OH, COOH, or a halogen

b) C₁₋₆ carboxy or alkoxy group, or

c) R¹ and R² comprise a substituent of the formula —O(CH₂)₃₋₅, wherein R¹ and R², together with the carbon atoms to which they are bonded, comprises a ring where at least one hydrogen atom thereof is optionally substituted with a halogen.

R³ is: a) (W)ₘ—Y-(Z)ₙ, wherein

W is a C₅₋₁₂ straight or branched (preferably 1S′CH₃, 2R′CH₃ dimethyl) alkyl, alkenyl, alkynyl, group, or mixture thereof, optionally substituted with at least one halogen,

Y is a bond, O, S, SO, SO₂, CO, NH, N(C₁₋₆ alkyl), or NCS,

Z is: i) a C₅₋₁₂ alkyl, alkenyl, alkynyl, group, or mixture thereof, optionally substituted with at least one halogen, optionally substituted with a terminal aromatic ring,

ii) CN₁₋₃, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different, or

iii) a phenyl or benzyl group, optionally substituted with halo, C₁₋₆ alkyl, C₁₋₆ alkoxy, C₁₋₆ alkylthio, CN, CF₃, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different, and wherein

m and n are the same or different, and each is either 0 or 1,

b) a C₅₋₁₂ alkyl or haloalkyl group, optionally substituted with a terminal aromatic ring, CN₁₋₃, NCS, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different, or

c) a C₅₋₁₂ alkene or alkyne group, optionally substituted with a halogen, dithiolene, terminal aromatic ring, CN₁₋₃, NCS, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different;

R⁶ and R^{6′} together form ═O or ═S, or each is independently selected from the group consisting of:

a) hydrogen,

b) C₁₋₆ alkoxy, C₁₋₆ alkylthio, C₁₋₆ alkyl, or C₁₋₆ haloalkyl,

c) CN,

d) CO₂H,

e) CO₂—C₁₋₄ alkyl,

f) C(Y)(Z)-OH,

g) C(Y)(Z)-O—C₁₋₄ alkyl, and

h) C₁₋₆ alkyl-CO₂—Y,

wherein Y and Z are each independently H or C₁₋₆ alkyl,

R⁷ is: a) hydroxy or lactone,

b) halo,

c) C₁₋₆ alkoxy, C₁₋₆ alkylthio, C₁₋₆ alkyl, or C₁₋₆ haloalkyl,

d) CN,

e) N₃,

f) CO₂H,

g) CO₂—C₁₋₄ alkyl,

h) C(Y)(Z)-OH,

i) C(Y)(Z)-O—C₁₋₄ alkyl,

j) C₁₋₆ alkyl-CO₂—Y, or

k)═O or ═S,

wherein Y and Z are each independently H or C₁₋₆ alkyl;

Q is: a) O or S, or

b) N—W, wherein W is:

i) hydrogen,

ii) C₁₋₆ alkoxyalkyl, C₁₋₆ alkyl, or C₁₋₆ haloalkyl

iii) OC₁₋₆ alkyl, or OC₁₋₆ haloalkyl,

iv) CN,

v) C₁₋₆ alkyl,

vi) C(Y)(Z)C₁₋₄ alkyl, or

vii) C₁₋₆ alkyl-CO₂-Z,

wherein Y and Z are each independently H or C₁₋₆ alkyl.

In on preferred embodiment R¹ in Formula II preferably is not OH, as it is in the natural cannabinol and tetrahydrocannabinol compounds. Rather, preferably R¹ in Formula II is H, O—C₁₋₄ alkyl (more preferably methoxy) or a hemi ester of succinic acid, malonic acid or the alaninate ester of alanine and salts thereof. In another preferred embodiment, R¹ and R² together comprise a substituent of the formula —O(CH₂)₃₋₅—, wherein R¹ and R², together with the carbon atoms to which they are bonded, comprise a ring where at least one hydrogen atom thereof is optionally substituted with a halogen (e.g., an O,2 propano ring). Furthermore, where R² Formula II is a halogen, preferably it is iodo. Preferably, R⁶ and R⁶ together form ═O or each are methyl, ethyl, or methoxy.

While R⁷ can be at any of positions 7-10 of ring C, preferably it is at position 9 of the ring, and preferably it is electronegative (e.g., COOH, halogen, β-hydroxy, or lactone.), and to enhance activity, it can be substituted with either a lactone or a β-hydroxy group.

Ring C in Formula II can be any of the following (the dashed lines representing a double bond at either the Δ6a-10a, Δ8-9, or Δ9-10 position):

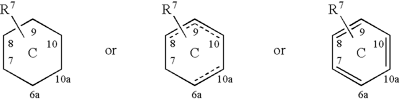

However, preferably the ring is aromatic. In such compounds, R⁷ preferably is electronegative and more preferably is on C9. Furthermore, R¹ preferably is other than OH and preferably is deoxy, an ester, or an ether. Exemplary cannabinol derivative compounds include:

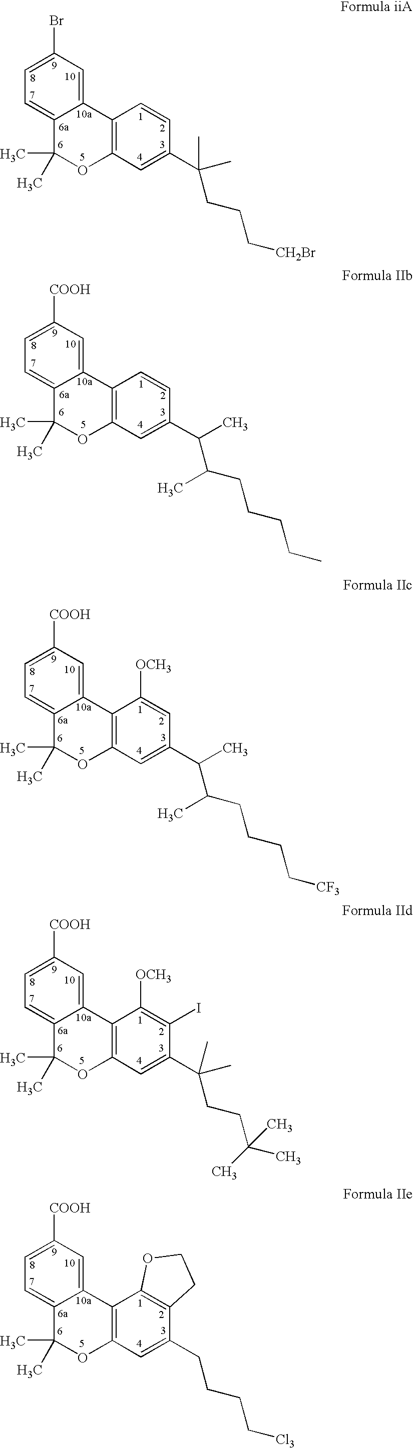

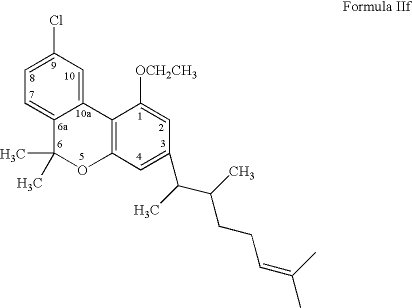

Many compounds according to Formula II are well known, and others can be manufactured in accordance with published methods (see, for example, International Patent Application WO99/20268 (Burstein), and U.S. Pat. No. 2,509,386 (Adams), U.S. Pat. No. 3,799,946 (Loev), U.S. Pat. No. 3,856,821 (Loev), U.S. Pat. No. 3,897,306 (Vidic et al.), U.S. Pat. No. 4,064,009 (Fukada et al.), U.S. Pat. No. 4,087,545 (Archer et al.), U.S. Pat. No. 4,142,139 (Bindra), U.S. Pat. No. 4,309,545 (Johnson), U.S. Pat. No. 4,599,327 (Nógrádi et al.), U.S. Pat. No. U.S. Pat. No. 4,833,073 (McNally et al.), U.S. Pat. No. 4,876,276 (Mechoulan et al.), U.S. Pat. No. 4,973,603 (Burstein), U.S. Pat. No. 5,338,753 (Burstein et al.), U.S. Pat. No. 5,389,375 (ElSohly), U.S. Pat. No. 5,440,052 (Makriyannis et al.), U.S. Pat. No. 5,605,906 (Lau), and U.S. Pat. No. 5,635,530 (Mechoulam et al.); and Charalambous et al., Pharm. Biochem. Behav., 40, 509-12 (191), Gareau et al., Bioorg. Med. Chem. Lett., 6(2), 189-94 (1996), Griffin et al., Br. J. Pharmacol., 126, 1575-8.4 (1999), Huffman et al., Bioorg. Med. Chem. Lett., 6, 2281-88 (1998), Lemberger et al., Clin. Pharmacol. Ther., 18(6), 720-26 (1975), Loev et al., J. Med. Chem., 16(11), 1200-06 9 (1973), Loev et al., J. Med. Chem., 17(11), 1234-35 (1974), Martin et al., Pharm. Biochem. Behav., 46, 295-301 (1993), Papahatjis et al., J. Med. Chem., 41(7), 1195-1200 (1998), Pars et al., J. Med. Chem., 19(4), 445-53 (1976), Pertwee et al., Pharmacol. Ther., 74(2), 129-80 (1997), Razdan et al., J. Med. Chem., 19(4), 454-60 (1976), Razdan, Pharmacol. Reviews, 38(2) 75-149 (1980), Reggio et al., J. Med Chem., 40(20), 3312-18 (1997), Reggio et al., Life Sci., 56(23/24), 2025-32 (1995), (Ross et al., Br. J. Pharmacol., 126, 665-72 (1999), Thomas et al., J. Pharm. Exp. Ther., 285(1), 285-92 (1998), Wiley et al., J. Pharm. Exp. Ther., 285(1), 995-1004 (1998), Winn et al., J. Med. Chem., 19(4), 461-71 (1976), and Xie et al., J. Med. Chem., 41, 167-74 (1998) ).

In the preferred embodiment wherein ring C of Formula II is aromatic, such compounds additionally can be manufactured by aromatizing an appropriate tetrahydrocannabinol (THC) derivative molecule by known methods (see, e.g., Adams et al., J. Am. Chem. Soc., 62, 23401 (1940); Ghosh et al., J. Chem. Soc., 1393 (1940); and Adams et al., J. Am. Chem. Soc., 70, 664 (1948)). For example, aromatization of such compounds can occur by heating the compound with sulfur at about 238-240° C., under a nitrogen atmosphere, for about 4 hours (Rhee et al., J. Med. Chem., 40(20), 3228-33 (1997)). Other suitable methods include aromatization using a catalyst (e.g., palladium on carbon) or a chemical dehydrogenating agent (e.g., 2,3-dichloro-5,6-dicyanoquinone) (see, for example, U.S. Pat. No. 3,799,946 (Loev)).

In other embodiments at least one compound for delivery to the blood product can be cannabidiol or a derivative thereof having the following formula:

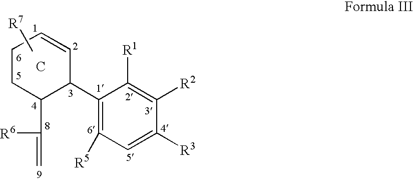

wherein:

R¹ is: a) H,

b) a C₁₋₄ alkyl group or ester thereof,

c) COOH,

d) OH,

e) a O—C₁₋₅ alkyl (preferably OCH₃) or alkanoyl, optionally substituted by mono- or di-methylamino or ethylamino groups,

f) a O—CO—C₃₋₁₀ alkyl group containing a carboxyl or amino group,

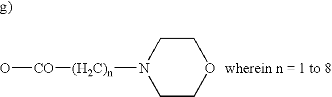

h) a p-aminobenzyl group or a C₁₋₇ aminoalkyl group or an organic or mineral acid addition salt thereof, an isocyanate or isothiocyanate derivative of the p-aminobenzyl or aminoalkyl group, a carboxyl terminated derivative of the aminoalkyl group having from 1 to 7 additional carbon atoms or a salt thereof, and an activated derivative of the carboxyl terminated derivative;

i) R¹ and R² comprise a substituent of the formula —O(CH₂)₃₋₅, wherein R¹ and R², together with the carbon atoms to which they are bonded, comprises a ring where at least one hydrogen atom thereof is optionally substituted with a halogen;

j) a lactone (e.g., COCOH); or

k) CH(CH₃)CO₂H or —OCOCH₃

R² is: a) H, OH, COOH, or a halogen

b) C₁₋₆ carboxy or alkoxy group, or

c) R¹ and R² comprise a substituent of the formula —O(CH₂)₃₋₅, wherein R¹ and R², together with the carbon atoms to which they are bonded, comprises a ring where at least one hydrogen atom thereof is optionally substituted with a halogen.

R³ is: a) (W)ₘ—Y-(Z)ₙ, wherein

W is a C₅₋₁₂ straight or branched (preferably IS′CH₃, 2R′CH₃ dimethyl) alkyl, alkenyl, alkynyl, group, or mixture thereof, optionally substituted with at least one halogen,

Y is a bond, O, S, SO, SO₂, CO, NH, N(C₁₋₆ alkyl), or NCS,

Z is: i) a C₅₋₁₂ alkyl, alkenyl, alkynyl, group, or mixture thereof, optionally substituted with at least one halogen, optionally substituted with a terminal aromatic ring,

ii) CN₁₋₃, CO₂H, or CO₂C₁₋₄ alkyl, CONH₂, CONHC₁₋₄ alkyl, or CON(C₁₋₄alkyl)₂, wherein each C₁₋₄ alkyl on the amide nitrogen can be the same or different, or

iii) a phenyl or benzyl group, optionally substituted with halo, C₁₋₆ alkyl, C₁₋₆ alkoxy, C₁₋₆ alkylthio, CN, CF₃, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different, and wherein

m and n are the same or different, and each is either 0 or 1,

b) a C₅₋₁₂ alkyl or haloalkyl group, optionally substituted with a terminal aromatic ring, CN₁₋₃, NCS, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different, or

c) a C₅₋₁₂ alkene or alkyne group, optionally substituted with a halogen, dithiolene, terminal aromatic ring, CN₁₋₃, NCS, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different;

R⁵ is a)H

b) a C₁₋₄ alkyl group

c) COOH

d) OH, or

e) a O—C₁₋₅ alkyl (ether) or alkanoyl, optionally substituted with at least one mono- or di-methylamino or ethylamino group;

R⁶ is:

a) hydrogen,

b) C₁₋₆ alkoxy, C₁₋₆ alkylthio, C₁₋₆ alkyl, or C₁₋₁₆ haloalkyl,

c) CN,

d) CO₂H,

e) CO₂—C₁₋₄ alkyl,

f) C(Y)(Z)-OH,

g) C(Y)(Z)-O—C₁₋₄ alkyl, or

h) C₁₋₆ alkyl-CO₂—Y,

wherein Y and Z are each independently H or C₁₋₆ alkyl,

R⁷ is: a) hydroxy or lactone,

b) halo,

c) C₁₋₆ alkoxy, C₁₋₆ alkylthio, C₁₋₆ alkyl, C₁₋₆ carboxy, or C₁₋₆ haloalkyl,

d) CN,

e) N₃,

f) CO₂H,

g) CO₂—C₁₋₄ alkyl,

h) C(Y)(Z)-OH,

i) C(Y)(Z)-O—C₁₋₄ alkyl,

j) C₁₋₆ alkyl-CO₂—Y, or

k) ═O or ═S,

wherein Y and Z are each independently H or C₁₋₆ alkyl, and wherein

R⁷ can be at any of positions 1, 2, 5, or 6 of ring C.

In addition to having the indicated substituents, R³ in any of formulas I-III preferably is:

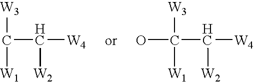

wherein W₁ is H, methyl, or ethyl, wherein W₂ and W₃ are each independently H or methyl, wherein at least one of W₁, W₂, and W₃ is other than H and/or halogenated, and wherein W₄ is a C₁₋₄ alkyl or haloalkyl, optionally substituted with an aromatic ring. Preferably, R³ is a branched C₆₋₁₂ alkyl group containing at least one double bond (more preferably at position C₄-C₁₀), and preferably the chain has an odd number of carbon atoms. More preferably, R³ is terminally branched or contains a terminal double bond, and the invention provides compounds according to Formulas I-V having such substituents. More preferably, R³ preferably is dimethylheptyl (DMH) (e.g., 1′,1′ DMH or 1′R, 2′S DMH), dimethylhexyl, or dimethylpentyl. For example, R³ can be a di- tri- or tetramethylpentyl, -hexyl, or -heptyl, etc., chain (e.g., 1,1,5-trimethylhexyl, 1,1,5,5-tetramethylhexyl, or 1,1,5-trimethyl-hept-4-enyl). In some instances, the R³ substituent can have bulky terminal moieties, for example, methyl, dimethyl, (CH₂)₁₋₆—CON(CH₃)₂, or C₆₋₁₂ haloalkyl with halogenated terminal carbon atoms (preferably bromine).

In the context of this invention, halogenated alkanes, alkenes, and alkynes can have any number of halogen substitutions. In a preferred embodiment, the halogenated alkane, alkene, or alkyne has at least one halogen on a terminal carbon atom (e.g., CX₁₋₃, wherein X is halogen). Alkyl groups (as well as alkenes and alkynes) can be straight chain or branched. Moreover, the compounds can exist as a single stereoisomer or a mixture of stereoisomers (e.g., a racemic mixture), or a single geometric isomer (e.g., E, Z, cis or trans) or a mixture of geometric isomers, all of which are within the scope of the invention. A particularly preferred compound for use in the inventive method is 2-Methyl-5-(1,1,5-trimethylhexyl)resorcinol (referred to hereinafter as IG-10).

In carrying out the inventive method, the cannabinoid or resorcinolic compound (or combinations of such compounds) is delivered into the blood product under conditions for it to inhibit or attenuate the aggregation and/or activation of platelets within the blood product. Generally, in performing the inventive method the cannabinoid, or resorcinolic compound is first formulated into a composition which is then introduced into the blood product. In this context, the invention also pertains to the use of a cannabinoid, or resorcinolic compound to prepare a composition suitable for inhibiting the aggregation and/or activation of blood platelets, as well as to such compositions, many of which are discussed below.

Where the blood product is in vitro the cannabinoid or resorcinolic compound can be admixed into the blood product. Typically, the compound is first formulated into an appropriately buffered solution (e.g., a physiologically-compatible saline solution), which is then mixed into the blood product. To effectively inhibit platelet aggregation within such blood products, typically a concentration of about 2×10⁻³ M to about 10×10⁻⁵ M should be employed (e.g., between about 0.1 mg/ml and about 4.0 mg/ml or even between about 1.0 mg/ml and about 2.5 mg/ml).

Desirably, the compound inhibits COX-1 and thromboxane synthase, as well as platelet aggregation induced by arachidonic acid but does not inhibit COX-2, all of which can be measured by standard methods. Without being bound by any particular theory, it is believed that selective inhibition of COX-1 will not only reduce thromboxane A2 synthesis in platelets but also PGE2 which is believed to potently reverse the antiaggretory effects of prostacyclin and prostaglandin D2 on human platelets. It is believed that a compound that possesses both COX-1 and thromboxane synthase inhibition would go further towards inhibition of aggregation of platelets than existing drugs. Moreover, preservation of endothelial COX-2 production of prostacyclin is further desirable because it is believed that such activity will preserve the production of prostacyclin and its activity in inhibiting platelet aggregation, promoting vasodilatation, and clot dissolution.

In this regard, the invention provides a method for inhibiting (COX-1) within a cell by exposing the cell to at least one cannabinoid or resorcinolic compound under conditions sufficient to inhibit COX-1 within the cell. A preferred cell for treatment in accordance with this aspect of the invention is a blood platelet cell, but the method can be used to treat any desired cell that exhibits COX-1 activity. Furthermore, it is desirable for the method also to inhibit thromboxane synthase in a cell, which can be the same cell or a different cell as that assayed for the inhibition of COX-1. It should be noted that it is not necessary for the method to result in complete inactivity of COX-1 or thromboxane synthase for the inventive method to be effective (although, this is desirable); indeed, it is sufficient for the method to significantly decrease the activity of these enzymes within the cell(s), which can be assayed using standard methods. Desirably, the method does not appreciably inhibit the activity of COX-2 in cells, particularly not in endothelial cells. While any suitable compound can be used in the inventive method, particularly preferred compounds for use in the inventive method are resorcinol derivatives, for example as set forth above. In this regard, 2-methyl-5-(1,1,5-trimethylhexyl)resorcinol can be employed effectively in conjunction with this aspect of the invention.

For in vivo use, the cannabinoid, or resorcinolic compound is desirably formulated into a pharmacologically-acceptable (i.e., pharmaceutically- or physiologically-acceptable) composition including a suitable carrier, and optionally other inactive or active ingredients. Such compositions are suitable for delivery by a variety of commonly-employed routes of delivery, such as, for example, buccal, sublingual, dermal, intraocular, intraotical, pulmonary, transdermal, intralymphatic, intratumor, intracavitary, intranasal, subcutaneous, implantable, inhalable, intradermal, rectal, vaginal, transmucosal, intramuscular, intravenous and intraarticular routes, among many others. Depending on the desired manner of application, the composition can include adjuvants, bile salts, biodegradable polymers and co-polymers, buffers, chelating agents, colorants, diluents, emollients, emulsifiers, enzyme inhibitors, hydrogels, hydrophilic agents, lipoproteins and other fatty acid derivatives, liposomes and other micelles, microporous membranes, mucoadhesives, neutral and hydrophobic polymers and co-polymers, particulate systems, perfumes, salt forming acids and bases, semi-permeable membranes, single or multiple enteric coatings, solvents (e.g., alcohols, dimethyl sulfoxide (DMSO), etc.), surfactants, viral envelope proteins, or other ingredients.

In one of its forms, a pharmacologically-acceptable can be an inhalable formulation comprising an aerosol of liquid or solid particles, such as are known in the art. Application of the composition via inhalation can treat bronchial conditions associated with inflammation (e.g., the common cold (rhinovirus), influenza, cystic fibrosis, etc.). This formulation can further comprise additional agents such as preservatives, antioxidants, flavoring agents, volatile oils, buffering agents, dispersants, surfactants, and the like, as are known in the art. Such formulation can also be provided with an inhalant, or in the inhalant, either in unit form or in a form which permits its repetitive use.

A pharmacologically-acceptable composition can also be a topical formulation (e.g., ointment, cream, lotion, paste, gel, spray, aerosol oil, etc.), wherein the carrier is a diluent for the agent suitable for topical delivery, e.g., petrolatum, lanoline, polyethylene glycols, alcohols and the like, optionally including trans-dermal enhancers. In the topical formulation, the carrier may be in a form suitable for formulating creams, gels, ointments, sprays, aerosols, patches, solutions, suspensions and emulsions.

A pharmacologically-acceptable composition can also be formulated for oral delivery, for example in the form of capsules, cachets, lozenges, tablets, powder, granules, solutions, suspensions, emulsions, essential oils (particularly hemp seed oil), etc. Such formulations typically include aqueous or non-aqueous liquid solutions and suspensions (e.g., oil-in-water or water-in-oil emulsions). Such oral formulations typically are encased in an enteric coating. Examples of oral formulations are buccal or sub-lingual formulation comprising lozenges which can also comprise flavoring agents and other known ingredients, or pastilles which can also comprise an inert base containing, for example, gelatin, glycerin, sucrose, acacia, and other ingredients and fillers as is known to the practitioner.

A pharmacologically-acceptable composition can also be a parenteral formulation, such as injectable solutions and suspensions. Typically, such formulations also comprise agents such as antioxidants, buffers, anti-bacterial agents, other anti-viral agents such as direct acting inhibitors of replication, and solutes which render the solution or suspension isotonic with the blood of an intended recipient. The solutions or suspensions are typically sterile aqueous or non-aqueous injectable solutions or suspensions, and can also comprise suspending agents and thickening agents. This formulation is generally provided in a sealed ampule or vial.

A pharmacologically-acceptable composition can also be a slow release formulation, which, when administered or applied to a subject, is capable of releasing a desired amount of the compound(s) over a pre-determined period of time. Alternatively, the composition can be a transdermal formulation, in which the carrier is suitable for facilitating the transdermal delivery of the agent. Examples are aqueous and alcoholic solutions, DMSO, oily solutions and suspensions, and oil-in-water or water-in-oil emulsions. A transdermal formulation can also be an iontophoretic transdermal formulation, in which typically the carrier can be an aqueous and/or alcoholic solution, an oily solution or suspension and an oil-in-water and water-in-oil emulsion. This formulation can further comprise a transdermal transport promoting agent, and be provided in the form of a kit with a transdermal delivery device, preferably an iontophoretic delivery device, many variations of which are known in the art.

Additional formulations of a pharmacologically-acceptable composition include, but are not limited to an implantable capsule or cartridge (e.g., for tissue implantation), a patch, an implant, or a suppository (e.g., for rectal or transmucosal administration). Typically, the composition will be distributed, either to physicians or to patients, in an administration kit, and the invention provides such a kit. Typically, such kits include, in separate containers, an administration device (e.g., syringes and needles, inhalators, pills, suppositories, transdermal delivery devices, etc) and a plurality of unit dosages of the composition as described above. In some kits, the composition can be preformulated. Other kits include separate ingredients for formulating the composition. The kit can additionally comprise a carrier or diluent, a case, and instructions for formulating the composition (if applicable) and for employing the appropriate administration device.

In carrying out the inventive method, the composition can be delivered to a patient in any amount and over any time course suitable for producing the desired therapeutic effect, and one of skill in the art will be able to determine an acceptable dosing schedule. Typically, the composition is delivered to a patient between 1 and about 6 times a day, if not continuously through transdermal or time release formulations. However, in some applications, it is appropriate to administer the composition less often. Generally each dose is between about 2 mg/m³ to about 1000 mg/m³, and more preferably about 0.01 mg/kg/day, about 1 mg/kg/day, such as about 10 ng/kg/day to about 10 mg/kg/day, and can be up to about 100 mg/kg/day (e.g., about 250 mg/kg/day). Moreover, the dosage amount and schedule can be reduced as a patient responds favorably to treatment and/or if any toxic side effects are noted.

In addition to employing a compound such as formulas I-III set forth herein, a pharmacologically-acceptable composition including the resorcinol derivative or cannabinoid derivative can be adjunctively employed as well. For example, the method can include the adjunctive administration of antineoplastics, antitumor agents, antibiotics, antifungals, antivirals (particularly antiretroviral compounds), antihelminthic, and antiparasitic compounds. Exemplary antiviral agents suitable for adjunctive use in the inventive method include abacavir, azidothymidine cidofovir, delavirdine mesylate, didanosine, dideoxycytidine, efavirenz, foscarnet, ganciclovir, indinavir sulfate, lamivudine, nelfinavir mesylate, nevirapine, ritonavir, saquinavir, saquinavir mesylate, stavudine, zalcitabine, etc. In treating tumors or neoplastic growths, suitable adjunctive compounds can include anthracycline antibiotics (such as doxorubicin, daunorubicin, carinomycin, N-acetyladriamycin, rubidazone, 5-imidodaunomycin, and N-acetyldaunomycin, and epirubicin) and plant alkaloids (such as vincristine, vinblastine, etoposide, ellipticine and camptothecin), paclitaxel and docetaxol, mitotane, cisplatin, phenesterine, etc. Anti-inflammatory therapeutic agents suitable for adjunctive use in the present invention include steroids and non-steroidal anti-inflammatory compounds, (such as prednisone, methyl-prednisolone, paramethazone; 11-fludrocortisol or fluorocortisone, triamciniolone, betamethasone and dexamethasone, ibuprofen, piroxicam, beclomethasone; methotrexate, azaribine, etretinate, anthralin, psoralins); salicylates (such as aspirin; and immunosuppresant agents such as cyclosporine). Additional pharmacologic agents suitable for adjunctive use in the inventive method include anesthetics (such as methoxyflurane, isoflurane, enflurane, halothane, and benzocaine); antiulceratives (such as cimetidine); antiseizure medications (such as barbituates; azothioprine (an immunosuppressant and antirheumatic agent); and muscle relaxants (such as dantrolene and diazepam). Moreover, the method can be employed in conjunction with specific antibody therapies or steroid therapies in treating autoimmune diseases. Other pharmacologically-active agents that can be adjunctively employed in conjunction with the composition include other constituents of natural marijuana having antimicrobial or anti-inflammatory activities (e.g., cannabigerol and its derivatives, cannabichromine and its derivatives, cannabinolic acid and its derivatives, cannabidiolic acid and its derivatives, terpenoids, flavanoids (e.g., cannflavin), etc.).

The composition can include biologically active agents, such as lymphokines or cytokines, anti-inflammatory, anti-bacterial, anti-viral, anti-fungal, anti-parasitic, anti-metabolic, anti-inflammatory, vasoactive, anti-neoplastic, bronchoacting, local anesthetic, immunomodulating, enzymatic, hormonal, growth promoting and regenerating agents, as well as neurotransmitters, and cell receptor proteins and ligands, among many other agents. Examples of other biological agents are analgesics (such as acetominophen, anilerdine, aspirin, buprenorphine, butabital, butorphanol, choline salicylate, codeine, dezocine, diclofenac, diflunisal, dihydrocodeine, elcatonin, etodolac, fenoprofen, hydrocodone, hydromorphone, ibuprofen, ketoprofen, ketorolac, levorphanol, magnesium salicylate, meclofenamate, mefenamic acid, meperidine, methadone, methotrimeprazine, morphine, nalbuphine, naproxen, opium, oxycodone, oxymorphone, pentazocine, phenobarbital, propoxyphene, salsalate, sodium salicylate, tramadol and narcotic analgesics in addition to those listed above). Anti-anxiety agents are also useful including alprazolam, bromazepam, buspirone, chlordiazepoxide, chlormezanone, clorazepate, diazepam, halazepam, hydroxyzine, ketaszolam, lorazepam, meprobamate, oxazepam and prazepam, among others. Other biologically-active agents include anti-anxiety agents associated with mental depression, such as chlordiazepoxide, amitriptyline, loxapine, maprotiline, and perphenazine, among others. Examples of other active ingredients include anti-inflammatory agents such as non-rheumatic aspirin, choline salicylate, diclofenac, diflunisal, etodolac, fenoprofen, floctafenine, flurbiprofen, ibuprofen, indomethacin, ketoprofen, lidomide, magnesium salicylate, meclofenamate, mefenamic acid, nabumetone, naproxen, oxaprozin, phenylbutazone, piroxicam, salsalate, sodium salicylate, sulindac, tenoxicam, tiaprofenic acid, thalidomide, linomide, and tolmetin, as well as anti-inflammatories for ocular treatment (such as diclofenac, flurbiprofen, indomethacin, ketorolac, and rimexolone (generally for post-operative treatment)), and anti-inflammatories for non-infectious nasal applications (such as beclomethaxone, budesonide, dexamethasone, flunisolide, triamcinolone, and the like); soporifics (anti-insomnia/sleep inducing agents) such as those utilized for treatment of insomnia, including alprazolaam, bromazepam, diazepam, diphenhydramine, doxylamine, estazolam, flurazepam, halazepam, ketazolam, lorazepam, nitrazepam, prazepam quazepam, temazepam, triazolam, zolpidem and sopiclone, among others; sedatives including diphenhydramine, hydroxyzine, methotrimeprazine, promethazine, propofol, melatonin, trimeprazine, and the like; sedatives and agents used for treatment of petit mal seizures and tremors, among other conditions, such as amitriptyline HCl; chlordiazepoxide, amobarbital; secobarbital, aprobarbital, butabarbital, ethchlorvynol, glutethimide, L-tryptophan, mephobarbital, methohexital sodium salt, midazolam HCl, oxazepam, pentobarbital Na, Phenobarbital, secobarbital sodium salt, thiamylal sodium, and many others. Other active compounds can include agents used in the treatment of head trauma (brain injury/ischemia), such as enadoline HCl (eg., for treatment of severe head injury), cytoprotective agents, and agents for the treatment of menopause, menopausal symptoms (treatment), e.g., ergotamine, belladonna alkaloids and phenobarbital, for the treatment of menopausal vasomotor symptoms, e.g., clonidine, conjugated estrogens and medroxyprogesterone, estradiol, estradiol cypionate, estradiol valerate, estrogens, conjugated estrogens, esterified estrone, estropipate, and ethinyl estradiol. Examples of agents for treatment of pre menstrual syndrome (PMS) are progesterone, progestin, gonadotrophic releasing hormone, oral contraceptives, danazol, luprolide acetate, vitamin B6; agents for treatment of emotional/psychiatric treatments such as tricyclic antidepressants including amitriptyline HCl (Elavil), amitriptyline HCl, perphenazine (Triavil) and doxepin HCl (Sinequan). Examples of tranquilizers, anti-depressants and anti-anxiety agents are diazepam (Valium), lorazepam (Ativan), alprazolam (Xanax), SSRI's (selective Seratonin reuptake inhibitors), fluoxetine HCl (Prozac), sertaline HCl (Zoloft), paroxetine HCl (Paxil), fluvoxamine maleate (Luvox) venlafaxine HCl (Effexor), serotonin, serotonin agonists (Fenfluramine); antibiotics (e.g., fluoroquinolones and tetracycline); antihistamines; catabolic steroids; and vasoactive agents (e.g., beta-blockers and pentoxiphylline (Trental)). Other compounds include cannabinoid, s such as CT-3 and HU-210.

### EXAMPLES

While one of skill in the art is fully able to practice the instant invention upon reading the foregoing detailed description, the following examples will help elucidate some of its features. Of course, as these examples are presented for purely illustrative purposes, they should not be used to construe the scope of the invention in a limited manner, but rather should be seen as expanding upon the foregoing description of the invention as a whole.

### Example 1

This example demonstrates the synthesis of a compound according to Formula I.

A mixture of 2,6-dimethoxyphenol (73.4 g, 0.48 mole), 2,6-dimethyl-2-heptanol (69.0 g, 0.48 mole) and methanesulfonic acid (95 mL) was stirred at 50° C. for 3 h and then at room temperature overnight. The mixture was poured over ice-water (600 mL) with stirring. The mixture was extracted with CH₂Cl₂ (2×200 mL). The extracts were washed with water, saturated aqueous NaHCO₃, saturated aqueous sodium chloride solution and dried over anhydrous Na₂SO₄. The solution was concentrated under reduced pressure to obtain the product as an oil (130 g, 96%). Analysis of this substance (MS (FAB) m/z 281 (MH)⁺; ¹H NMR (CDCl₃) δ 0.80 (d, 6H), 1.0-1.1 (m, 4H), 1.27 (s, 6H), 1.40-1.60 (m, 3H), 3.89 (s, 6H), 5.36 (s, 1H), 6.54 (s, 2H)) revealed it to be 4-(1,1,5-trimethylhexyl)-2,6-dimethoxyphenol (referred to hereinafter as IG-02):

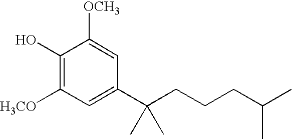

### Example 2

This example demonstrates the synthesis of a compound according to Formula I.

A solution of crude 4-(1,1,5-trimethylhexyl)-2,6-dimethoxyphenol from Example 1 (130 g, 0.46 mole) in dry CCl₄ (100 mL) was cooled in ice-bath and diethyl phosphite (70 mL, 0.54 mole) was added. To the stirred mixture triethylamine (75 mL, 0.54 mole) was added dropwise at such a rate as to maintain the temperature of the reaction mixture below 10° C. The reaction mixture was stirred in the ice-bath for 2 h and at room temperature overnight. The mixture was then diluted with CH₂Cl₂ (200 mL), washed with water, 4N aqueous NaOH (100 mL), 1N aqueous HCl (125 mL), water and saturated aqueous sodium chloride solution. The extracts were dried over anhydrous Na₂SO₄ and concentrated under reduced pressure. The crude product was purified by chromatography over a column of silica using cyclohexane:EtOAc (7:1 to 3:1 gradient) as the eluent to obtain 103 g (54%) of the product as a colorless waxy oil. Analysis of this substance (MS (FAB) m/z 417 (MH)⁺. ¹H NMR (CDCl₃) δ 0.81 (d, 6H), 1.0-1.1 (m, 4H), 1.26 (s, 6H), 1.35-1.6 (m, 9H), 3.86 (s, 6H), 4.25-4.38 (m, 4H), 6.53 (s, 2H)) revealed it to be 4-(1,1,5-trimethylhexyl)-2,6-dimethoxyphenyl diethyl phosphate:

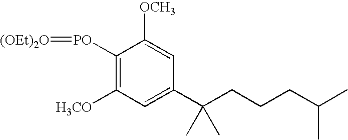

### Example 3

This example demonstrates the synthesis of a compound according to Formula I.

A solution of 4-(1,1,5-trimethylhexyl)-2,6-dimethoxyphenyl diethyl phosphate from Example 2 (82 g, 0.197 mole) in Et₂O (175 mL) and THF (35 mL) was added slowly to liquid ammonia (450 mL) contained in a 3-neck vessel fitted with mechanical stirrer, thermometer, dry ice condenser and a pressure equalizing addition funnel while adding small freshly cut pieces of lithium wire (2.8 g, 0.40 g-atom) at such a rate as to maintain a blue color. The reaction mixture was stirred further for an hour and then quenched by the addition of saturated aqueous NH₄Cl (22 mL). Ether (220 mL) was added and the ammonia was allowed to evaporate overnight. The residue was treated with water (220 mL). The layers were separated and the ether layer was washed with 4N NaOH (200 mL), water (2×200 mL) and saturated aqueous sodium chloride solution. The organic extracts were dried (MgSO₄) and concentrated under reduced pressure. The crude product was purified by chromatography over a column of silica using cyclohexane:EtOAc (95:5) as the eluent to obtain 43 g (83%) of the product as a colorless oil. Analysis of this substance (MS (FAB) m/z 265 (MH)⁺; ¹H NMR (CDCl₃) δ 0.80 (d, 6H), 1.00-1.10 (m, 4H), 1.26 (s, 6H), 1.4-1.6 (m, 3H), 3.79 (s, 6H), 6.30 (m, 1H), 6.49 (m, 2H)) revealed it to be 4-(1,1,5-trimethylhexyl)-2,6-dimethoxybenzene (referred to hereinafter as IG-03):

### Example 4

This example demonstrates the synthesis of a compound according to Formula I.

A solution of 4-(1,1,5-trimethylhexyl)-2,6-dimethoxybenzene from Example 3 (10 g, 0.038 mole) in anhydrous CH₂Cl₂ (100 mL) was cooled in ice-bath and was treated dropwise with a solution of boron tribromide in CH₂Cl₂ (100 mL of 1M solution, 0.10 mole) over a period of 1 h. The mixture was stirred in the cold bath for 2 h and then at room temperature overnight. The reaction mixture was cooled in ice-bath and cautiously treated with water (100 mL). The resulting mixture was diluted with CH₂Cl₂ (100 mL) and treated with half-saturated aqueous sodium bicarbonate solution. The layers were separated, the organic layer was concentrated to half volume under reduced pressure and extracted with 2N aqueous NaOH (2×75 mL). The aqueous alkaline extract was cooled and acidified to pH 3.0 with 1N aqueous HCl. The acidified mixture was extracted with Et₂O (2×100 mL). The ether layer was washed with saturated aqueous sodium chloride solution, dried over anhydrous MgSO₄ and concentrated under reduced pressure. The crude product thus obtained was purified by chromatography over a column of silica using cyclohexane:EtOAc (8:1 to 4:1 gradient) as the eluent to obtain 8.0 g (90%) of the product as colorless crystalline solid. Analysis of this substance (Mp 95-96° C. MS (FAB) m/z 237 (MH)⁺; ¹HNMR (CDCl₃) δ 0.80 (d, 6H), 1.00-1.10 (m, 4H), 1.23 (s, 6H), 1.40-1.58 (m, 3H), 4.65 (s, 2H), 6.17 (m, 1H), 6.38 (m, 2H)) revealed it to be 5-(1,1,5-trimethylhexyl) resorcinol (referred to hereinafter as IG-01):

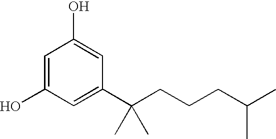

### Example 5

This example demonstrates the synthesis of a compound according to Formula I.

A solution of 4-(1,1,5-trimethylhexyl) resorcinol from Example 4 (2 g, 0.0076 mole) in anhydrous CH₂Cl₂ (10 mL) was cooled in ice-bath and was treated dropwise with a solution of boron tribromide in CH₂Cl₂ (2.6 mL of 1M solution, 0.0026 mole). The mixture was stirred in the cold bath for 2 h and then at room temperature overnight. The mixture was cooled in ice-bath and cautiously treated with water (10 mL) followed by saturated aqueous sodium bicarbonate (5 mL). The organic layer was separated, dried over MgSO₄ and concentrated under reduced pressure. The residue was purified by chromatography over a column of silica using cyclohexane:EtOAc (8:1 to 4:1 gradient) as the eluent to obtain 0.364 g (19%) of the product as a colorless oil. Analysis of this substance (MS (FAB) m/z 251 (MH)⁺; ¹H NMR (CDCl₃) δ 0.80 (d, 6H), 1.00-1.10 (m, 4H), 1.24 (s, 6H), 1.4-1.6 (m, 3H), 3.78 (s, 3H), 4.67 (s, 1H), 6.23 (m, 1H), 6.40 (m, 1H), 6.47 (m, 1H)) revealed it to be 3-methoxy-5-(1,1,5-trimethylhexyl)phenol (referred to hereinafter as IG-04):

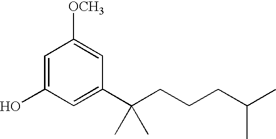

### Example 6

This example demonstrates the synthesis of a compound according to Formula I.

To solution of crude 4-(1,1,5-trimethylhexyl)-2,6-dimethoxyphenol from Example 1 (0.19 g, 0.68 mmol) in dry THF (6 mL) was added iodomethane (0.78 g, 5.4 mmol). The mixture was treated with 60% dispersion of sodium hydride in mineral oil (0.06 g, 1.5 mmol) under nitrogen atmosphere. The mixture was stirred at room temperature for 24 h and then concentrated under reduced pressure. The residue was treated with ether (20 mL). Water (5 mL) was added cautiously. The layers were separated, the ether layer was washed with water (5 mL), dried (MgSO₄) and concentrated under reduced pressure. The crude product was purified by chromatography over a column of sillica using cyclohexane/EtOAc 6:1 as the eluent to obtain 0.17 g (85%) of the product. Analysis of this substance (MS (FAB) m/z 295 (MH)⁺. ¹H NMR (CDCl₃) δ 0.81 (d, 6H), 1.0-1.2 (m, 4H), 1.28 (s, 6H), 1.40-1.60 (m, 3H), 3.84 (s, 3H), 3.87 (s, 6H), 6.53 (s, 2H)) revealed it to be 1-(1,1,5-Trimethylhexyl)-3,4,5-trimethoxybenzene (referred to hereinafter as IG-07):

### Example 7

This example demonstrates the synthesis of a compound according to Formula II.

A solution of 5-(1,1,5-trimethylhexyl) resorcinol (0.472 g, 2 mmol), p-menth-2-ene-1,8-diol (0.30 g, 2.1 mmol) and p-toluenesulfonic acid (0.084 g) in dry benzene (25 mL) was refluxed under a Dean-Stark trap for 4 h. The mixture was cooled to room temperature and treated with saturated aqueous sodium bicarbonate (25 mL). The layers were separated. The aqueous layer was extracted with benzene. The combined organic extracts were dried (MgSO₄) and concentrated under reduced pressure. The crude product was chromatographed over a column of silica gel using cyclohexane/EtOAc 95:5 as the eluent to obtain 0.22 g (30%) of the product. Analysis of the product (MS (FAB) m/z 371 (MH)+. 1H NMR (CDCl3) δ 0.80 (d, 6H), 1.00-1.10 (m, 4H), 1.11 (s, 3H), 1.21 (s, 6H), 1.39 (s, 3H), 1.4-1.52 (m, 3H), 1.71 (s, 3H), 1.75-1.95 (m, 3H), 2.1-2.2 (m, 1H), 2.62-2.73 (m, 1H), 3.12-3.25 (m, 1H), 4.61 (s, 1H), 5.4-5.5 (m, 1H), 6.23 (s, 1H), 6.39 (s, 1H)) revealed it to be 3-Norpentyl-3-(1,1,5-trimethylhexyl)-Δ 8-tetrahydrocannabinol (referred to hereinafter as IG-09):

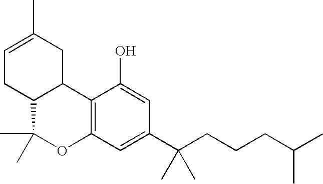

### Example 8

This example demonstrates the synthesis of a compound according to Formula I.

A solution of 4-(1,1,5-trimethylhexyl)-2,6-dimethoxyphenol (10 g, 35.7 mmol) in dry pyridine (70 mL) was cooled to 0° C. To the stirred solution was added dropwise trifluoromethanesulfonic anhydride (11 g, 39 mmol). After the addition was complete, the reaction mixture was allowed to warm to room temperature and stir at room temperature overnight under argon. To the mixture was added an additional quantity of trifluromethanesulfonic anhydride (1.7 g, 6 mmol) and stirred for 2 h at room temperature. The mixture was concentrated under reduced pressure to remove most of the pyridine. The residue was treated with cold water (100 mL) and extracted with CH₂Cl₂ (3×50 mL). The organic extracts were washed with 1N HCl and brine, dried and concentrated under reduced pressure to obtain an orange syrup (14 g, 95%). The triflate thus obtained was used as such in the next step.

A mixture of the above triflate (10 g, 23.3 mmol), anhydrous lithium chloride (8.3 g, 196 mmol), triphenylphosphine (3.83 g, 14.6 mmol) and dichlorobis(triphenylphosphine)palladium (II) (1.8 g, 2.6 mmol) in anhydrous DMF (110 mL) was placed in a stainless steel pressure vessel under an atmosphere of nitrogen. To this mixture was added tetramethyltin (10 g, 56 mmol) and a few mg of 2,6-di-tert-butyl-4-methylphenol. The mixture was heated in an oil bath at 120° C. for 24 h. An additional quantity of tetramethyltin (5.5 g, 19 mmol) and a few crystals of 2,6-di-tert-butyl-4-methylphenol were added and the mixture was heated at 130° C. for 24 h. The mixture was cooled to room temperature and was filtered through a pad of celite to remove the palladium catalyst. The filtrate was concentrated under reduced pressure to ¼ the volume and filtered to remove yellow solid. The filtrate was further concentrated to near dryness. The residue was dissolved in CH₂Cl₂ (200 mL) and washed successively with 1.5 N HCl (5×100 mL), saturated aqueous potassium fluoride (5×50 mL), and brine. The organic layer was dried (MgSO₄) and concentrated under reduced pressure to obtain dark oil. This was purified by chromatography over a column of silica using cyclohexane/CH₂Cl₂ gradient (97:3 to 90:10) to obtain 1.82 g (27%) of the dimethoxy methyl compound. This product was utilized as such in the next step.

A solution of the above dimethoxy compound (1 g, 3.6 mmol) in CH₂Cl₂ (20 mL) was cooled to 0° C. and treated dropwise with 1M solution of BBr₃ in CH₂Cl₂ (7.2 mL, 7.2 mmol). The mixture was stirred in the cold bath for 2 h and then at room temperature overnight. The reaction mixture was cooled in an ice bath and diluted with half-saturated aqueous sodium bicarbonate solution (20 mL). The mixture was diluted with CH₂Cl₂ (25 mL), and the layers were separated. The organic extracts were dried (MgSO₄) and the solvent was removed under reduced pressure to obtain a beige solid which was purified by chromatography over a column of silica using cyclohexane/EtOAc 95:5 as the eluent to obtain 0.41 g (46%) of the product. Analysis of the product (Mp 145-147o C. MS (FAB) m/z 251 (MH)+. 1H NMR (CDCl3) d 0.80 (d, 6H), 1.00-1.10 (m, 4H), 1.21 (s, 6H), 1.40-1.55 (m, 3H), 2.11 (s, 3H), 2.07 (s, 2H), 6.37 (s, 2H)) revealed it to be 2-Methyl-5-(1,1,5-trimethylhexyl)resorcinol (referred to hereinafter as IG-10):

### Example 10

This example demonstrates the synthesis of a compound according to Formula II.

A mixture of 3-norpentyl-3-(1,1,5-trimethylhexyl)-Δ8-tetrahydrocannabinol (1.4 g, 3.8 mmol) and elemental sulfur (0.3 g, 0.5 mmol) was placed in a test tube and heated in a sand bath at 240-260° C. for 3 h. The crude product was purified by chromatography over a column of silica using cyclohexane/EtOAc 97:3 as the eluent to obtain 0.7 g (51%) of the product. Analysis of the product (MS (FAB) rm/z 367 (MH)+. 1H NMR (CDCl3) δ 0.79 (d, 6H), 1.00-1.11 (m, 4H), 1.25 (s, 6H), 1.38-1.58 (m, 3H), 1.60 (s, 6H), 2.39 (s, 3H), 5.09 (s, 1H), 6.41 (s, 1H), 6.56 (s, 1H), 7.05 (d, 1H), 7.15 (d, 1H), 8.16 (s, 1H)) revealed it to be 3-Norpentyl-3-(1,1,5-trimethylhexyl)cannabinol (referred to hereinafter as IG-11):

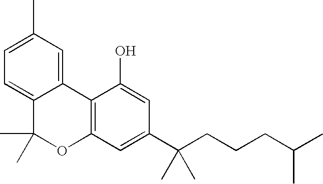

### Example 11

This example demonstrates the use of compounds as described herein to inhibit the aggregation of blood platelets.

Test compounds referred to above were evaluated for inhibition of platelet aggregation induced by adenosine diphosphate, arachidonic acid, phorbol ester and Platelet Activating Factor (PAF) at 30 mM. The following methods were employed in this analysis, and reference compounds for the respective assays are indicated in Appendix 1:

Adenosine Diphosphate, Platelet Aggregation

Venous blood obtained from male or female New Zealand derived albino rabbits weighing 2.5-3 kg was mixed with one-tenth volume of trisodium citrate (0.13 M) and centrifuged at room temperature for 10 min at 220 g. Test substance (30 EM)-induced aggregation of the supernatant platelet rich plasma by 50 percent or more (>50%) more relative to 1.2 μM adenosine diphosphate control response at 37° C. as mearsured by an optical aggregometer, indicates possible ADP receptor agonist activity.

At a test substance concentration where no significant agonist activity is seen, ability to reduce the adenosine diphosphate-induced maximum non-reversible aggregation response by 50 percent or more (≧50%) was indicative of ADP receptor antagonist activity.

Arachidonic Acid, Platelet Aggregation

Venous blood obtained from male or female New Zealand derived albino rabbits weighting 2.5-3 kg was mixed with one-tenth volume of trisodeium citrate (0.13M) and centrifuged at room temperature for 10 min at 220 g. Test substance (30 μM)-induced aggregation of the supernatant platelet rich plasma by 50 percent or more (≧50%) within 5 min, relative to 100 μM arachidonic acid response at 37° C. as measured by an optical aggregometer indicates possible agonist activity.

At a test substance concentration where no significant agonist activity is seen, ability to reduce the arachidonic acid-induced maximum non-reversible aggregation response by 50 percent or more (>50%) was indicative of antagonist activity.

Phorbol Ester, Platelet Aggregation

Venous blood obtained from male or female New Zealand derived albino rabbits weighing 2.5-3 kg is mixed with one-tenth volume of trisodium citrate (0.13 M) and centrifuged at room temperature for 10 min at 220 g. Test substance (30 μM)-induced aggregation of the supernatant platelet rich plasma by 50 percent or more (≧50%) within 5 min, relative to control phorbol myristate acetate (PMA, 0.5 μM) response at 37° C. as measured by an optical aggregometer, indicates possible phorbol ester receptor agonist activity.

At a test substance concentration where no significant agonist activity is seen, ability to reduce the PMA (0.5 μM)-induced maximum non-reversible aggregation response by 50 percent or more (>50%) indicates phorbol ester receptor antagonist activity.

Platelet Activating Factor

Venous blood obtained from male or female New Zealand derived albino rabbits weighing 2.5-3 kg is mixed with one-tenth volume of trisodium citrate (0.13 M) and centrifuged at room temperture for 10 min at 220 g. Test Substance (30 μM)-induced aggregation of the supernatant platelet rich plasma by 50 percent or more (≧50%) within 5 min, relative to control 5 nM platelet activating factor-acether (PAF-acether) response at 37% C as measured by an optical aggregometer, indicates possible PAF receptor agonist activity.

At a test substance concentration where no significant agonist activity is seen, ability to reduce the PAF-acether-induced maximum non-reversible aggregation response by 50 percent or more (≧50%) indicates PAF receptor antagonist activity.

The results of these experiments are indicated in Appendix 1. Significant inhibition (100%) was observed for IG-10 versus arachidonic acid-induced platelet aggregation. These data are consistent with the ability of IG-10 to inhibit platelet activation and/or aggregation.

### Example 12

This example demonstrates the use of compounds as described herein to inhibit the aggregation of blood platelets.

Test compounds (IG-1 through IG-11) were evaluated for their ability to inhibit cyclooxygenase COX-1, cyclooxygenase COX-2, and thromboxane synthase. In these assays, in addition to the designations set forth above, IG-05 refers to resorcinol and IG-06 refers to orcinol.

To assess COX-1 inhibition, test compound and/or vehicle was incubated with human platelets (5×10⁷/well) for 15 minutes at5 37° C. Calcium ionophore A23187 (10 μM) was added to induce the arachidonic acid cascade. After another 15 minutes incubation at 37° C., PGE₂ levels in the supernatant were quantiated using the Amersham EIA kit. Compounds were screened at 10 μM. Results were considered significant if a test compound exhibited ≧50% maximal stimulation or inhibition.

To assess COX-1 inhibition, cyclooxygenase-2 (human recombinant) isolated from Spodoptera frugiperda was used. Test compound and/or vehicle was pre-incubated with 0.11 U cyclooxygenase-2, 1 mM reduced GSH, 500 μM phenol and 1 μM hematin for 15 minutes at 37° C. The reaction was initiated by addition of 0.3 μM arachidonic acid as substrate in Tris-HCl pH 7.7 and terminated after 5 minutes incubation at 37° C. by addition of 1N HCl. Following centrifugation, substrate conversion to PGE2 was measured by an Amersham EIA kit. Compounds are screened at 10 μM. Results were considered significant if a test compound exhibited ≧50% maximal stimulation or inhibition.

To assess Thromboxane A₂ synthase, thromboxane A₂ synthase isolated from a microsomal fraction of rabbit platelets by conventional centrifugation was used. The test compound and/or vehicle was incubated with 1:200 dilution of thromboxane A₂ synthase and 5 ng prostaglandin G2 as substrate in Tris buffer pH 7.5 for 30 minutes at 37° C. The thromboxane A₂ formed is immediately converted to thromboxane B2, which was quantitated by a radioimmunoassay. Compounds are screened at 100 μM. Results were considered significant if a test compound exhibited ≧50% maximal stimulation or inhibition.

The results of these assays, and reference compounds employed in them, are presented in Appendix 2. The data indicate that IG-2, IG-6, and IG-10 are potent inhibitors of COX-1 and that IG-10 and IG-11 significantly inhibit thromboxane synthase. These data are consistent with the ability of IG-10 to inhibit platelet activation and/or aggregation, and also COX-1 and thromboxane synthase but not COX-2.

### Incorporation by Reference

All sources (e.g., inventor's certificates, patent applications, patents, printed publications, repository accessions or records, utility models, world-wide web pages, and the like) referred to or cited anywhere in this document or in any drawing, Sequence Listing, or Statement filed concurrently herewith are hereby incorporated into and made part of this specification by such reference thereto.

### Guide to Interpretation

The foregoing is an integrated description of the invention as a whole, not merely of any particular element of facet thereof. The description describes “preferred embodiments” of this invention, including the best mode known to the inventors for carrying it out. Of course, upon reading the foregoing description, variations of those preferred embodiments will become obvious to those of ordinary skill in the art. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law.

As used in the foregoing description and in the following claims, singular indicators (e.g., “a” or “one”) include the plural, unless otherwise indicated. Recitation of a range of discontinuous values is intended to serve as a shorthand method of referring individually to each separate value falling within the range, and each separate value is incorporated into the specification as if it were individually listed. As regards the claims in particular, the term “consisting essentially of” indicates that unlisted ingredients or steps that do not materially affect the basic and novel properties of the invention can be employed in addition to the specifically recited ingredients or steps. In contrast, the terms “comprising” or “having” indicate that any ingredients or steps can be present in addition to those recited. The term “consisting of” indicates that only the recited ingredients or steps are present, but does not foreclose the possibility that equivalents of the ingredients or steps can substitute for those specifically recited.

### Appendix 1, Page 1

TISSUE ASSAYS

| | | | | | | CRI- | | | | |
|---|---|---|---|---|---|---|---|---|---|---|
| COMPOUND CODE | PT NUMBER | BATCH* | TISSUE, SPECIES | n | CONC. | TERIA | RESP. | AG. | ANT. | R |
| Diphosphate, | | | | | | | | | | |
| Platelet Aggregation | | | | | | | | | | |
| IG-7 | 1010321 | 27737 | platelet rich plasma, rabbit | 2 | 30 μM | ≧50% | | 0% | 11% | |
| IG-8 | 1010322 | 27737 | platelet rich plasma, rabbit | 2 | 30 μM | ≧50% | | 0% | 8% | |
| IG-9 | 1010323 | 27737 | platelet rich plasma, rabbit | 2 | 30 μM | ≧50% | | 0% | 7% | |
| IG-10 | 1010324 | 27737 | platelet rich plasma, rabbit | 2 | 30 μM | ≧50% | | 0% | 17% | |
| IG-11 | 1010325 | 27737 | platelet rich plasma, rabbit | 2 | 30 μM | ≧50% | | 0% | 0% | |
| 412510 | | | | | | | | | | |
| Arachidonic | | | | | | | | | | |
| Acid, Platelet | | | | | | | | | | |
| Aggregation | | | | | | | | | | |
| IG-7 | 1010321 | 27731 | platelet rich plasma, rabbit | 2 | 30 μM | ≧50% | | 0% | 0% | |
| IG-8 | 1010322 | 27731 | platelet rich plasma, rabbit | 2 | 30 μM | ≧50% | | 0% | 0% | |
| IG-9 | 1010323 | 27731 | platelet rich plasma, rabbit | 2 | 30 μM | ≧50% | | 0% | 0% | |
| ♦ IG-10 | 1010324 | 27731 | platelet rich plasma, rabbit | 2 | 30 μM | ≧50% | | 0% | 100% | |
| IG-11 | 1010325 | 27731 | platelet rich plasma, rabbit | 2 | 30 μM | ≧50% | | 0% | 0% | |
| 416000 | | | | | | | | | | |
| Cannabinoid CB₁ | | | | | | | | | | |
| ♦ IG-7 | 1010321 | 27098 | vas deferens, mouse | 2 | 30 μM | ≧50% | | −106% | 100% | |
| ♦ IG-8 | 1010322 | 27098 | vas deferens, mouse | 2 | 30 μM | ≧50% | | −34% | 82% | |
| ♦ IG-11 | 1010325 | 27098 | vas deferens, mouse | 2 | 30 μM | ≧50% | | 102% | ND | |
| 461500 Phorbol | | | | | | | | | | |
| Ester, Platelet | | | | | | | | | | |
| Aggregation | | | | | | | | | | |
| IG-7 | 1010321 | 27699 | platelet rich plasma, rabbit | 2 | 30 μM | ≧50% | | 0% | 9% | |
| IG-8 | 1010322 | 27699 | platelet rich plasma, rabbit | 2 | 30 μM | ≧50% | | 0% | 8% | |
| IG-9 | 1010323 | 27699 | platelet rich plasma, rabbit | 2 | 30 μM | ≧50% | | 0% | 4% | |
| IG-10 | 1010324 | 27699 | platelet rich plasma, rabbit | 2 | 30 μM | ≧50% | | 0% | 9% | |
| IG-11 | 1010325 | 27699 | platelet rich plasma, rabbit | 2 | 30 μM | ≧50% | | 0% | 8% | |
| 463010 PAF, | | | | | | | | | | |
| Platelet Aggregation | | | | | | | | | | |
| IG-7 | 1010321 | 27927 | platelet rich plasma, rabbit | 2 | 30 μM | ≧50% | | 0% | 4% | |
| IG-8 | 1010322 | 27927 | platelet rich plasma, rabbit | 2 | 30 μM | ≧50% | | 0% | 8% | |
| IG-9 | 1010323 | 27927 | platelet rich plasma, rabbit | 2 | 30 μM | ≧50% | | 0% | 4% | |
| IG-10 | 1010324 | 27927 | platelet rich plasma, rabbit | 2 | 30 μM | ≧50% | | 0% | 6% | |
| IG-11 | 1010325 | 27927 | platelet rich plasma, rabbit | 2 | 30 μM | ≧50% | | 0% | 11% | |

*Batch: Represents compounds tested concurrently in the same assay(s).
♦ Denotes item meeting criteria for significance
Ag. = Agonist;
Ant. = Antagonist;
Resp. = Response;
ND = Assay Test Not Done;
R = Additional Comments

APPENDIX 1

| | | | REFERENCE | CONCURRENT | | |
|---|---|---|---|---|---|---|
| CAT. # | ASSAY NAME | TISSUE, SPECIES | COMPOUND | BATCH* | CONC. | RESP. |
| 404010 | Adenosine diPO₄, Platelet Aggregation - | platelet rich plasma, | ADP | 27737 | 1.2 μM | 100 |
| | Antagonist | rabbit | | | | |
| | | platelet rich plasma, | 2-Chloroadenosine | 27737 | 10 μM | 90 |
| | | rabbit | | | | |
| 412510 | Arachidonic Acid, Platelet | platelet rich plasma, | Arachidonic Acid | 27731 | 100 μM | 100 |
| | Aggregation - Agonist | rabbit | | | | |
| 412510 | Arachidonic Acid, Platelet | platelet rich plasma, | Indomethacin | 27731 | 0.3 μM | 100 |
| | Aggregation - Antagonist | rabbit | | | | |
| 416000 | Cannabinoid CB₁ - Agonist | vas deferens, mouse | Anandamide | 27098 | 0.1 μM | 100 |
| 463010 | PAF, Platelet Aggregation - Agonist | platelet rich plasma, | PAF | 27927 | 5 nM | 100 |
| | | rabbit | | | | |
| 463010 | PAF, Platelet Aggregation - Antagonist | platelet rich plasma, | WEB-2086 | 27927 | 0.3 μM | 97 |
| | | rabbit | | | | |
| 461500 | Phorbol Ester, Platelet Aggregation - | platelet rich plasma, | PMA | 27699 | 0.5 μM | 100 |
| | Agonist | rabbit | | | | |
| 461500 | Phorbol Ester, Platelet Aggregation - | platelet rich plasma, | Staurosporine | 27699 | 3 μM | 100 |
| | Antagonist | rabbit | | | | |

*Batch: Represents compounds tested concurrently in the same assay(s).

### Appendix 2, Page 2

APPENDIX 2

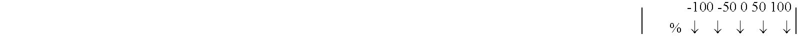

116010 Cyclooxygenase COX-1

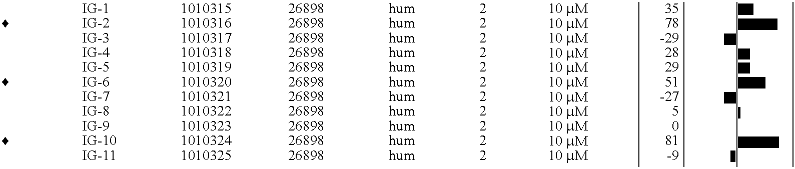

118010 Cyclooxygenase COX-2

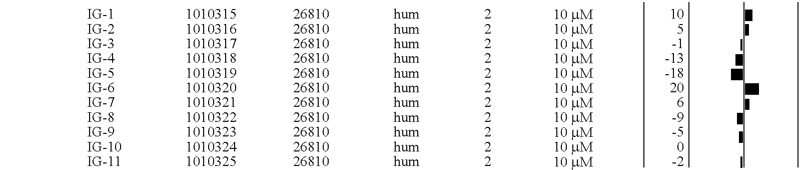

194000 Thrombaxane Synthetase

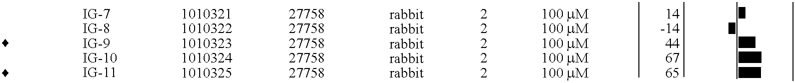

217010 Cannabinoid CB₁

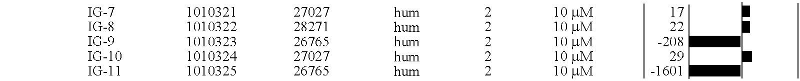

217100 Cannabinoid CB₂

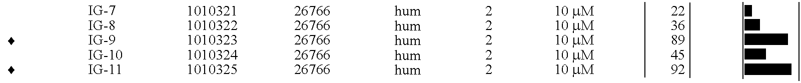

TISSUE ASSAYS

| | | | | | | CRI- | | | | |
|---|---|---|---|---|---|---|---|---|---|---|
| COMPOUND CODE | PT NUMBER | BATCH * | TISSUE, SPECIES | n | CONC. | TERIA | RESP. | AG. | ANT. | R |
| 404010 Adenosine | | | | | | | | | | |
| Diphosphate, Platelet | | | | | | | | | | |
| Aggregation | | | | | | | | | | |
| IG-7 | 1010321 | 27737 | platelet rich plasma, rabbit | 2 | 30 μM | ≧50% | | 0% | 11% | |
| IG-8 | 1010322 | 27737 | platelet rich plasma, rabbit | 2 | 30 μM | ≧50% | | 0% | 8% | |
| IG-9 | 1010323 | 27737 | platelet rich plasma, rabbit | 2 | 30 μM | ≧50% | | 0% | 7% | |
| IG-10 | 1010324 | 27737 | platelet rich plasma, rabbit | 2 | 30 μM | ≧50% | | 0% | 17% | |
| IG-11 | 1010325 | 27737 | platelet rich plasma, rabbit | 2 | 30 μM | ≧50% | | 0% | 0% | |
| 412510 Arachidonic | | | | | | | | | | |
| Acid, Platelet | | | | | | | | | | |
| Aggregation | | | | | | | | | | |
| IG-7 | 1010321 | 27731 | platelet rich plasma, rabbit | 2 | 30 μM | ≧50% | | 0% | 0% | |
| IG-8 | 1010322 | 27731 | platelet rich plasma, rabbit | 2 | 30 μM | ≧50% | | 0% | 0% | |
| IG-9 | 1010323 | 27731 | platelet rich plasma, rabbit | 2 | 30 μM | ≧50% | | 0% | 0% | |
| ♦ IG-10 | 1010324 | 27731 | platelet rich plasma, rabbit | 2 | 30 μM | ≧50% | | 0% | 100% | |
| IG-11 | 1010325 | 27731 | platelet rich plasma, rabbit | 2 | 30 μM | ≧50% | | 0% | 0% | |
| 416000 | | | | | | | | | | |
| Cannabinoid CB₁ | | | | | | | | | | |
| ♦ IG-7 | 1010321 | 27098 | vas deferens, mouse | 2 | 30 μM | ≧50% | | −106% | 100% | |
| ♦ IG-8 | 1010322 | 27098 | vas deferens, mouse | 2 | 30 μM | ≧50% | | −34% | 82% | |
| ♦ IG-11 | 1010325 | 27098 | vas deferens, mouse | 2 | 30 μM | ≧50% | | 102% | ND | |
| 461500 Phorbol | | | | | | | | | | |
| Ester, Platelet | | | | | | | | | | |
| Aggregation | | | | | | | | | | |
| IG-7 | 1010321 | 27699 | platelet rich plasma, rabbit | 2 | 30 μM | ≧50% | | 0% | 9% | |
| IG-8 | 1010322 | 27699 | platelet rich plasma, rabbit | 2 | 30 μM | ≧50% | | 0% | 8% | |
| IG-9 | 1010323 | 27699 | platelet rich plasma, rabbit | 2 | 30 μM | ≧50% | | 0% | 4% | |
| IG-10 | 1010324 | 27699 | platelet rich plasma, rabbit | 2 | 30 μM | ≧50% | | 0% | 9% | |
| IG-11 | 1010325 | 27699 | platelet rich plasma, rabbit | 2 | 30 μM | ≧50% | | 0% | 8% | |
| 463010 PAF, | | | | | | | | | | |
| Platelet Aggregation | | | | | | | | | | |
| IG-7 | 1010321 | 27927 | platelet rich plasma, rabbit | 2 | 30 μM | ≧50% | | 0% | 4% | |
| IG-8 | 1010322 | 27927 | platelet rich plasma, rabbit | 2 | 30 μM | ≧50% | | 0% | 8% | |
| IG-9 | 1010323 | 27927 | platelet rich plasma, rabbit | 2 | 30 μM | ≧50% | | 0% | 4% | |
| IG-10 | 1010324 | 27927 | platelet rich plasma, rabbit | 2 | 30 μM | ≧50% | | 0% | 6% | |
| IG-11 | 1010325 | 27927 | platelet rich plasma, rabbit | 2 | 30 μM | ≧50% | | 0% | 11% | |

| | | |
|---|---|---|
| REFERENCE | HISTORICAL | CONCURRENT MIC |

| CAT. # | ASSAY NAME | COMPOUND | IC₅₀ | Kᵢ | n_{H} | BATCH * | IC₅₀ |
|---|---|---|---|---|---|---|---|
| 116010 | Cyclooxygenase COX-1 | Indomethacin | 10 nM | | | 26898 | 0.011 μM |
| 118010 | Cyclooxygenase COX-2 | Nimesulide | 1 μM | | | 26810 | 2.21 μM |
| 194000 | Thromboxane Synthetase | Dazoxiben | 0.022 μM | | | 27758 | 0.061 μM |
| 217010 | Cannabinoid CB₁ | WIN-55,212-2 | 0.029 μM | 0.023 μM | 0.8 | 26765 | 0.044 μM |
| | | WIN-55,212-2 | 0.029 μM | 0.023 μM | 0.8 | 27027 | 0.025 μM |
| | | WIN-55,212-2 | 0.029 μM | 0.023 μM | 0.8 | 28271 | 0.053 μM |
| 217100 | Cannabinoid CB₂ | WIN-55,212-2 | 5.8 nM | 3.9 nM | 1.1 | 26766 | 0.012 μM |
| | | WIN-55,212-2 | 5.8 nM | 3.9 nM | 1.1 | 27028 | 7.57 nM |

* Batch: Represents compounds tested concurrently in the same assay(s).
♦ Denotes item meeting criteria for significance
† Results with ≧50% stimulation or inhibition are boldfaced. (Negative values correspond to stimulation of binding or enzyme
R = Additional Comments
hum = human
Ag. = Agonist;
Ant. = Antagonist;
Resp. = Response;
ND = Assay Test Not Done;

What is claimed is:

1. A method for attenuating the activation or aggregation of blood platelets within a blood product comprising introducing at least one cannabinoid or resorcinolic compound into the blood product under conditions sufficient to inhibit the aggregation of blood platelets within the blood product.

2. The method of claim 1, wherein the blood product is ex vivo.

3. The method of claim 2, wherein the blood product is within an organ or tissue.

4. The method of claim 1, wherein the blood product is whole blood.

5. The method of claim 1, wherein the blood product is in vivo.

6. The method of claim 1, wherein the compound is a resorcinol derivative.

7. The method of claim 6, wherein the resorcinol derivative is introduced into the blood product at a concentration of from about 10×10⁻⁵ M to about 2×10⁻³ M.

8. The method of claim 1, wherein the compound is 2-Methyl-5-(1,1,5-trimethylhexyl)resorcinol.

9. The method of claim 1, wherein the method attenuates the activation of blood platelets.

10. The method of claim 1, wherein the method prevents the activation of blood platelets.

11. The method of claim 1, wherein the method attenuates the aggregation of blood platelets.

12. The method of claim 1, wherein the method prevents the aggregation of blood platelets.

13. A method for inhibiting cyclooxygenase-1 (COX-1) within a cell or platelet, which comprises exposing the cell or platelet to at least one cannabinoid or resorcinolic compound under conditions sufficient to inhibit COX-1 within the cell or platelet.

14. The method of claim 13, which does not inhibit the activity of COX-2.

15. The method of claim 13, which further inhibits the activity of thromboxane synthase within the cell or platelet

16. The method of claim 13, wherein the compound is 2-methyl-5-(1,1,5-trimethylhexyl)resorcinol.

17. The method of claim 13, wherein the compound is a resorcinol derivative.

18. The method of claim 13, wherein the COX-1 is inhibited within a platelet.

19. The method of claim 13, wherein the COX-1 is inhibited within a cell.

20. The method of claim 13, wherein the cell or platelet is within an organ or tissue.

21. The method of claim 13, wherein the cell or platelet is within blood product

22. The method of claim 21, wherein the blood product is whole blood.

23. The method of claim 21, wherein the compound is introduced into the blood product at a concentration of from about 10×10⁻⁵ M to about 2×10⁻³ M.

24. The method of claim 21, wherein the compound is introduced into the blood product at a concentration of from about 0.1 mg/ml to about 4 mg/ml.

25. The method of claim 21, wherein the compound is introduced into the blood product at a concentration of from about 1 mg/ml to about 2.5 mg/ml.
